(19) United States
(12) Patent Application Publication
(10) Pub. No.: US 2011/0152836 A1
(43) Pub. Date: Jun. 23, 2011

(54) Method and Apparatus for Arterial and Venous Cannulation

(75) Inventors: James Riopelle, New Orleans, LA (US); Valeriy Kozmenko, Metairie, LA (US); Susheela Viswanathan, New Orleans, LA (US); Alan D. Kaye, River Ridge, LA (US)

(73) Assignee: Board of Supervisors of Louisiana State University and Agricultural and Mechanical College, Baton Rouge, LA (US)

(21) Appl. No.: 12/963,339

(22) Filed: Dec. 8, 2010

Related U.S. Application Data
(60) Provisional application No. 61/283,751, filed on Dec. 8, 2009.

(51) Int. Cl. A61M 25/06; A61M 25/09

(52) U.S. Cl. 604/510; 604/158; 604/163

(57) ABSTRACT

A method and apparatus for cannulating vessels and cavities are provided. Such an apparatus includes a y-adaptor having a front leg configured to hold a needle, a guide-wire leg configured to receive a guide-wire, and a syringe leg configured to hold a syringe for aspiration. Once the needle is inserted into the desired vessel or cavity, the syringe may be aspirated to determine if the insertion was successful. Then, a guide-wire may be pushed into the guide-wire leg and into the needle via the front leg of the apparatus. Once the guide-wire has reached the targeted vessel or cavity, a catheter or cannula may be positioned over the guide wire and advanced to the desired location.

### CROSS-REFERENCE TO RELATED APPLICATION

This application claims the benefit of U.S. Provisional Patent Application Ser. No. 61/283,751 filed on Dec. 8, 2009 which is expressly incorporated herein in its entirety by reference hereto.

### FIELD

The present invention relates to cannulation of vessels or cavities.

### BACKGROUND

The Seldinger technique is a cannulation procedure that has become widely adopted for angiography, arterial and central venous catheterization, insertion of chest drainage tubes, insertion of percutaneous feeding (e.g. gastrostomy) tubes, insertion of the leads for an artificial pacemaker or implantable cardioverter-defibrillator, and numerous other interventional medical procedures. The first step of the Seldinger technique is to puncture the vessel or cavity with a hollow needle. When fluid in the vessel or cavity is at, or minimally above, atmospheric pressure, a syringe is attached to the needle to permit the generation of subatmospheric (“negative”) pressure and to facilitate withdrawal of fluid (blood, urine, pleural fluid, etc.) from the vessel or cavity, thereby indicating to the practitioner that the vessel or cavity has been successfully entered. Once the hollow needle is in place, if the syringe is easily aspirated then it is clear the cavity has been entered; however, if substantial force is required to aspirate the syringe then it is clear that the vessel or cavity has not been entered. This technique is not necessary for vessels or cavities with substantial super-atmospheric pressure because the fluid from the vessel or cavity will spontaneously enter the needle giving confirmation to the individual performing the technique. Once it has been confirmed that the target fluid-containing cavity has been entered by the needle tip, the syringe is disconnected from the needle and a guide-wire is advanced through the needle into the vessel or cavity. The needle is then removed. The guide-wire is pushed through the vessel or cavity the desired distance into the target vessel or cavity. A cannula may then be inserted over the guide-wire and pushed through to the desired location. The guide-wire is then removed and the cannula is secured in the proper position.

A modification to the Seldinger technique has been used for particularly tortuous vessels and cavities. In this modification the guide-wire has a j-shaped-tip in order to permit its advancement through vessels and cavities with tight turns and valves without becoming caught or snagged. The use of a j-tip can be especially useful when advancing a catheter along the course of the often very tortuous external jugular vein. This modification has also been found to facilitate safe cannulation of all but the smallest arteries and veins.

Since the time that these techniques were initially developed, kits have been manufactured and sold to permit cannulation of vessels and body cavities. Many kits still follow the traditional Seldinger technique and sell components for each step of the process, yet some account for advancements to the Seldinger technique, including using a j-tip guide-wire instead of a straight-tip guide-wire, a three legged adapter for a guide-wire to be fed through the needle, and an apparatus that allows for a catheter to be placed over the needle, instead of through the needle.

Current cannulation systems suffer from numerous drawbacks. If the pressure within the target vessel or cavity is low (e.g. the external jugular vein of a dehydrated patient), many of these kits can permit air entrainment (air embolism) which, when it is carried to the heart and lungs, can compromise the function of these organs and may lead to death. Many current systems also fail to take into account an appropriate surface on the apparatus that can be comfortably and safely sutured to a patient. For example, the flexibility (elastic modulus) of the suture attachment portion of many cannula assemblies does not match that of the skin to which the assembly is being attached, the sutures from the assembly can “piston” in and out of the skin with each movement of the catheter. Such pistoning can increase the amount of cutaneous bacteria that enters the body. Many current systems also fail to consider any sort of integrated sterile protection for the guide-wire as it is fed into the apparatus. Unless scrupulous aseptic techniques on a large sterile field are used, the guide-wire can become contaminated from bacteria during the cannulation process.

Additionally, currently available cannulation systems do not incorporate the ideal geometry to facilitate guiding the guide-wire into the needle. Reported devices have a syringe leg parallel to the needle, and a guide-wire leg at an angle to the needle. However, since it is much more difficult for the guide-wire to bend than for air or liquid being aspirated to turn a corner it may be better to have the guide-wire—needle angle at zero degrees and the needle—syringe angle at greater than zero.

Moreover, many needles included in currently marketed vascular cannulation kits are not optimal for Seldinger vascular cannulation. The degree of sharpness (bevel angle) of needles included in most currently marketed central venous cannulation kits is insufficient to permit the needle tip to reliably enter a vein that it encounters. Instead, the relatively blunt (“venous bevel”) needle tip frequently compresses and flattens the vein causing the needle tip to pass through both walls of the vessel (a “double-wall puncture”) and into the underlying tissue. In the case of peripheral venous or arterial cannulation, this often results in the blood loss from the vessel into the surrounding tissues and failure to cannulate the vessel (“blowing the vein”), thus requiring additional attempts to cannulate alternative vessels (thereby inflicting additional discomfort on the patient). In the case of central venous cannulation, if the underlying tissue is the carotid artery (true in about twenty percent of cannula insertions into the internal jugular vein) this can produce a stroke with permanent brain damage. If the underlying tissue is lung (a major concern during cannulation of the subclavian vein), this can produce potentially life-threatening lung collapse requiring surgical insertion of a chest tube. The problem of currently marketed relatively blunt cannulating needles compressing rather than penetrating the lumens (internal fluid-containing portions) of veins is especially a problem in: central and peripheral veins with low internal pressure, which is present in dehydrated patients or those suffering from shock (e.g., due to great loss of blood); the veins of well-hydrated patients if the target vein cannot be occluded by tourniquet application (especially the external jugular vein); and veins that must be entered slowly because the needle is being guided into position using in-plane 2-dimensional ultrasound guidance (a very delicate process during which there is considerable difficulty maintaining the needle within the planet of the ultrasound visualization). To overcome the problem of blunt needles causing double-wall puncture, physicians must result to using strategies such as very rapid short needle advances.

Despite the risks set forth above, there are two likely reasons relatively blunt needles are nevertheless included in many current vascular access kits. 1) Blunt needles (i.e. with “venous bevels”) for intravenous infusions were originally left in veins for long periods of time to infuse fluids and drug solutions. In such situations, sharper, long-beveled needles would have increased the likelihood of vessel laceration and fluid extravasation (leakage), especially in a moving patient. and great sharpness only increased the likelihood of vessel laceration and needle tip displacement outside the vessel lumen. 2) Currently available very sharp (“standard” or “long-bevel”) needles have a very long distance from the needle tip to the most proximal (nearest to the needle hub or syringe connection point) portion of the needle opening, thus increasing the length of the needle opening relative to the vessel diameter and thereby potentially increasing the likelihood of the point of the needle penetrating the deeper vessel wall (a “double-wall puncture”) before fluid is withdrawn into the aspirating syringe, confirming lumen entry.

Apart from the issue of needle sharpness, the straight needles packaged with many commercial vascular access kits are not optimal for the puncture of certain veins, such as the external jugular vein, particularly when the presence of more protruberant surrounding tissue (e.g. the jawbone in the case of the external jugular vein), immediately adjacent to the usual puncture site forces the operator to choose a very steep angle of approach to the vein, further increasing the risk of a double-wall puncture. This problem can occur even during ultrasound-guided cannulation of deeper veins, especially small veins of the antecubital area in patients with increased subcutaneous tissue (fat). In the case of ultrasound-guided venous cannulation, the physician performing the cannulation is often forced to choose between slower advancement so that he/she can visualize the needle clearly throughout the process (entailing an increased probability of vein compression), and faster, unguided advancement without the benefit of ultrasound guidance increasing the risk of missing the vein entirely or advancing the needle too far, the needle tip traversing the vein rather than stopping within its lumen.

The inadequacy of many currently available venous cannulation kits is particularly obvious during cannulation of the veins of people who have undergone many hospitalizations (i.e. elderly or chronically ill patients) and therefore have few remaining veins suitable for cannulation (typically the external jugular veins and deeper arm veins visible only with ultrasound imaging). In fact, it is fairly common that, in such (especially non-obese) patients, the only peripheral vein still available for cannulation is the external jugular vein. When presently available venous cannulation kits do not permit cannulation of this vein, physicians must resort to the more dangerous central venous cannulation.

### SUMMARY

According to an exemplary embodiment of the present invention, an apparatus for cannulating a vessel or cavity is provided that includes a y-adapter having a front leg, a guide-wire leg opposite the front leg, and a syringe leg configured to receive a syringe for aspiration. A needle, which may be a straight beveled needle or a concavely beveled needle, is connected such that it protrudes outward from the front leg of the apparatus. Upon insertion of the needle into the targeted vessel or cavity, a user may aspirate a syringe connected to the syringe leg to determine if the vessel or cavity has been successfully entered. If the vessel or cavity has been successfully entered, a guide-wire may be pushed into the guide-wire leg, through the device and into the needle via the front leg, which may be tapered. Once the guide wire is in place in the targeted vessel or cavity, a catheter or cannula may be positioned over the guide wire and into the targeted vessel or cavity.

### BRIEF DESCRIPTION OF THE DRAWINGS

FIG. 1 is a profile view of an three legged apparatus for cannulation of vessels or cavities according to an embodiment of the present invention.

FIG. 2 is a profile view of a straight beveled needle tip and a progressively beveled needle tip according to an embodiment of the present invention.

FIG. 3 is an isometric view of a straight beveled needle tip and a progressively beveled needle tip according to an embodiment of the present invention.

FIG. 4 is a profile view of a leg of a cannulation device allowing entry of a guide-wire according to an embodiment of the present invention.

FIG. 5 is a profile view of a leg of a cannulation device allowing a needle and a catheter hub to be placed according to an embodiment of the present invention.

FIG. 6 is a profile view of a curved and progressively beveled needle with a tapered hub that may be attached to a cannulation device according to an embodiment of the present invention.

FIG. 7A-C are various views of plugs that seal a guide-wire cover to a cannulation device according to an exemplary embodiment of the present invention. FIG. 7A is a front view of an open plug. FIG. 7B is an isometric view of an open plug. FIG. 7C is an isometric view of a closed plug.

### DETAILED DESCRIPTION

The present invention resolves significant drawbacks present in currently available cannulation devices. Embodiments of the present invention provide a method and apparatus designed for vessel and cavity cannulation. The needle used in this apparatus may have at least the sharpness of a needle typically used to draw blood specimens for laboratory testing such that it can enter vessels and cavities of humans or animals with extreme ease. According to some embodiments, the apparatus may include a needle with a progressive bevel configured to cannulate vessels or cavities, and the needle may also be curved. The apparatus may also include a chamber with at least two openings: a first opening may lead to a curved needle with a small-angle or progressive bevel, and a second opening may lead to an entry for a guide-wire. The first opening may include a fitting configured to receive the needle and/or a removable attached catheter, which may be configured to fit over the needle. The apparatus may further include a needle hub, which may be tapered, configured to lead the needle and to fit on the first opening. The apparatus may also include a catheter hub configured to lead to the catheter and fit on the first opening. The catheter hub may contain a suture neck, which may contain suture holes and may only be large enough to provide for the suture holes. The apparatus may also include a flexible cover, such as a bag, to protect the guide wire from contaminants.

The apparatus may further include a second opening in the chamber that includes a seal configured to allow a guide wire to enter into the chamber. The guide wire may have a j-tip, and the seal may be fluid and/or air proof. The seal itself may include an o-ring and/or a valve. A third opening in the chamber for fitting a syringe may also be included. The syringe may be configured to receive a plurality of fluid volumes.

According to an embodiment of the disclosed invention, the device may also include a Y-chamber/Y-adapter having an anterior opening and two posterior openings. The anterior opening may have a male luer- or luer-lock type adapter that connects to a gently-curved needle over which is fitted an intravenous-type plastic cannula with female luer-type hub designed to ultimately be advanced into the lumen of the target blood vessel or other hollow organ (“catheter-over-needle” design). The catheter hub may include an indented “neck” to permit secure attachment of a skin suture or an elastomeric collar with side holes for attachment of one or two skin sutures. One posterior opening may be at a slight angle to the line of needle insertion with a female luer-lock type adapter for connection to an aspirating syringe. The second posterior opening may be in line with the line of needle insertion to serve as the entry port for a wire guide (either j-tip or straight-tip or with both types of tips and reversible). To this port designed to make an air-tight connection with a clear protective cover (bag) included to ensure the maintenance of guide wire sterility as it is manually advanced (or retracted) during the cannulation process. The bag not only fits snugly around the Y-chamber/Y-adapter port, but also may include an o-ring or other type of valve to create an air-tight seal around the wire guide.

The present invention also discloses a method for cannulating vessels or cavities utilizing an apparatus as described in the foregoing embodiments. According to an embodiment of the present invention, such a method may include the steps of: puncturing the vessel or cavity with the needle; passing the guide wire through the chamber and through the needle into the vessel or cavity to the desired point of interest; removing the catheter from the first opening; passing the catheter over the needle; passing the catheter over the guide wire to the point of interest; and removing the entire apparatus, except for the catheter, from the patient. Such a method may further involve attaching a syringe to the third opening of the apparatus, and the syringe may be aspirated after the vessel or cavity is breached. Further, in certain embodiments wherein the apparatus includes a catheter hub with a suture neck, the catheter hub may be stitched to the patient.

According to an exemplary embodiment of the present invention, a method may include the steps of: puncturing the vessel or cavity with a very sharp short-bevel (i.e. progressively-beveled) needle, such a bevel being created either by 2 curved secondary grinds or secondary and tertiary (and perhaps quaternary—each closer to the needle hub) grinds of progressively greater angle; attaching a syringe to the posterior opening of the apparatus, wherein the plunger of the syringe can be withdrawn after the needle is placed beneath the skin to create a subatmospheric pressure within the syringe barrel, facilitating withdrawal of fluid when the needle tip enters the vessel/organ lumen; additional fluid can be aspirated once some has been seen to enter the syringe chamber to provide additional confidence that it continues to lie within the vessel/organ lumen (i.e., has not been unintentionally moved); passing the guide wire (with either a j-tip or soft straight-tip) through the Y-chamber/Y-adapter and then through the needle into the target vessel or cavity; advancing the catheter over the needle so that its tip resides in the target lumen; and removing the entire apparatus, except for the catheter, from the patient. In certain embodiments, wherein the apparatus includes a catheter hub with a suture neck, the catheter hub may be stitched to the patient.

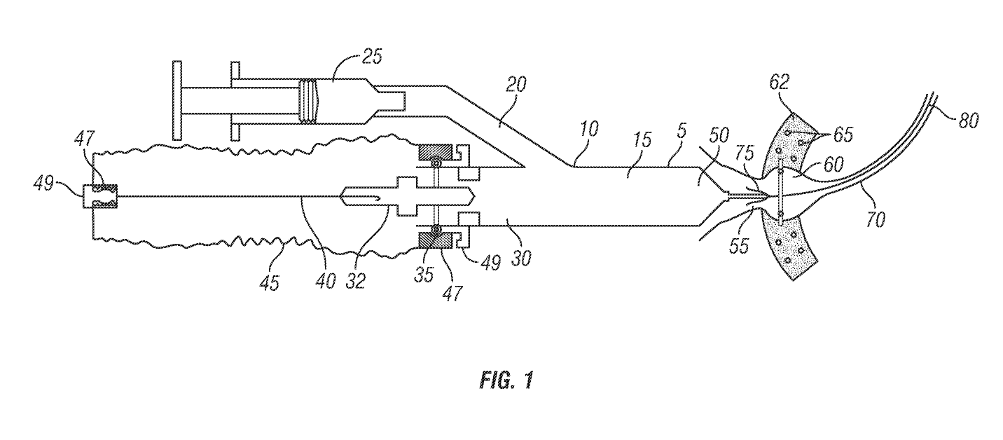

FIG. 1 shows a profile view of an embodiment of a cannulation device 5 for purposes including—but not limited to—administration of fluids or drugs, intravascular pressure monitoring, collection of blood samples for laboratory analysis, and insertion of a device (e.g., intravascular catheter, biliary catheter, dialysis catheter, pacemaker wire). According to an exemplary embodiment of the present invention, the main body of the device may include a Y-adapter 10 having a central cavity 15 and three legs: a syringe leg 20, a guide-wire leg 30, and a front leg 50. Syringe leg 20 may be configured to receive a syringe 25. Syringe 25 may be used if negative pressure is desired through aspiration. A practitioner may desire to use aspiration to confirm that a vessel or cavity has been entered. If the vessel or cavity contains fluid, even if under very low pressure, then the plunger of syringe 25 can be withdrawn from the barrel of syringe 25 and the fluid will be withdrawn. If the plunger is difficult to release then it is likely that the target vessel lumen or cavity has not been breached. However, if the vessel or cavity is under substantial super-atmospheric pressure (e.g., in the case of an artery), releasing the plunger of a syringe 25 may not be necessary to show that the vessel or cavity has been entered because the fluid from the vessel or cavity may spontaneously flow into syringe 25 through needle 80, thereby giving the desired confirmation to the practitioner. In fact, if the fluid within the vessel or organ lumen is known to be under very high pressure, certain embodiments may omit attachment of syringe 25. Syringe leg 20 may be parallel to guide-wire leg 30, however in certain embodiments syringe leg 20 is at an angle to guide-wire leg 30 to facilitate easier handling of device 5.

Device 5 may also include a guide-wire leg 30 of Y-adapter 10 that may allow a guide-wire 40 to pass through the main cavity 15 of Y-adapter 10. Guide-wire leg 30 may be in line with front leg 50, while syringe leg 20 may be at an angle to guide-wire leg 30 and front leg 50. In some embodiments, this angle may be about 45 degrees. This placement may allow for a straight, lower-friction path for a guide-wire 40 into a needle 80 (reducing the possibility of guide-wire hang-up or bending), and may provide the user of device 5 with more comfort in handling the device. This placement may allow a user to push a guide-wire 40 in from below, as opposed to from above, and may allow the user to ignore syringe 25 entirely after it may have been aspirated.

Surrounding a guide-wire 40 may be a protective cover or plastic bag 45 that may prevent contaminants from entering Y-adapter 10. In certain embodiments, protective cover 45 may be air-tight and/or sterile. Using a bag 45 may allow a physician or other practitioner to aseptically advance a guide-wire 40 into a Y-adapter 10 without use of sterile gloves, if desired. Additionally, a guide-wire 40 may have a j-tip on one end and a straight-tip on the other, both contained fully within the sterile chamber provided by bag 45. Each side of a bag 45 may have a plug 47 that may attach to Y-adapter 10. This allows a practitioner to choose which type of tip is best and attach the correct plug to a Y-adapter 10 without searching for a separate guide-wire 40. A plug 47 not attached to a Y-adapter 10, may seal onto itself, or, in other embodiments, may be sealed by a separate cover. To prevent fluid back-flow, in certain embodiments an o-ring 35 may operate as a seal between a plug 47 and Y-adapter 10. An o-ring 35 may also operate as a seal between a plug 47 and a separate cover. Note, however, that a valve for a wire-guide 32 may not necessarily be water-tight if bag 45 has a smaller volume than syringe 25. A catheter hub 55 may attach to Y-adapter 10 at front leg 50. Catheter hub 55 may contain a suture neck 60 and suture holes 65. These features may permit suturing of the cannula hub to skin once the cannula is within the target vessel/organ lumen. An elastomric suture connection to the cannula hub at the suture neck which includes wings and suture holes hub 60 may allow for minimal movement of a catheter 70 with respect to the peri-catheter skin of the patient. Elastomeric material 62 of flexibility (elastic modulus) similar to the skin to which it may be attached may reduce pistoning of the catheter in and out of the skin puncture site thus reducing entrainment of skin bacteria into the catheter track leading to the target vessel/organ lumen. Use of such an elastomeric connection between the skin and cannula may also provide more patient comfort. Further down an end of a Y-adapter 50, past a catheter hub 55, a needle's tapered hub 75 may connect to a Y-adapter 10. A tapered hub 75 may lead to a needle 80, which in certain embodiments be curved and concavely beveled. In the embodiment illustrated in FIG. 1, a catheter 70 covers a portion of needle 80. The cannulating needle's tapered hub 75 may make a tight connection with the lumen of catheter 40.

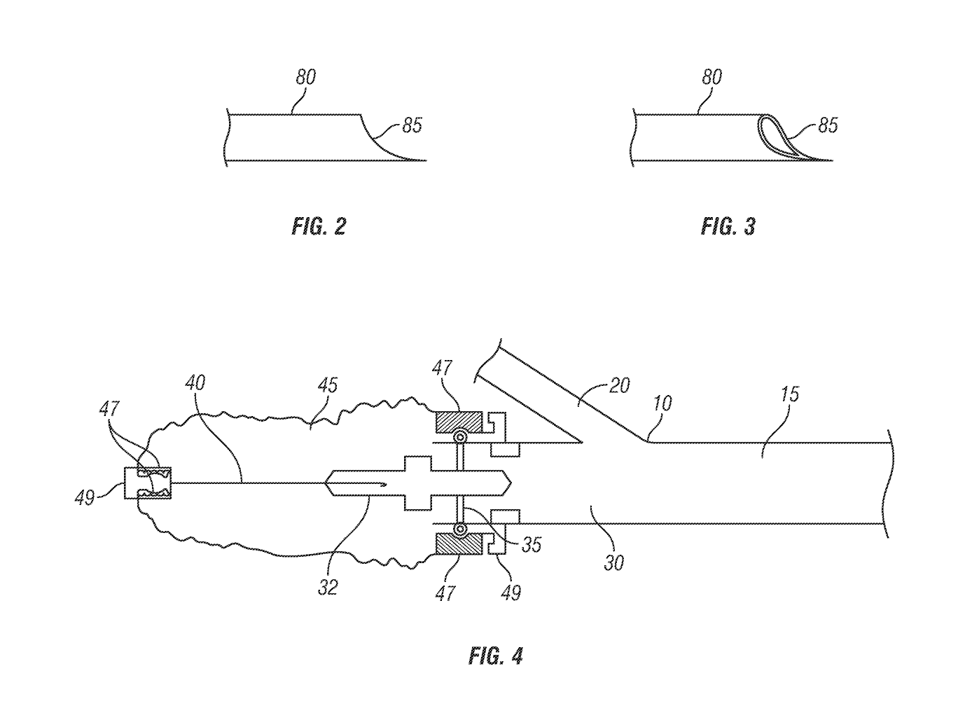

FIGS. 2 and 3 show a concavely beveled needle tip 85. A traditional straight beveled needle tip exists when the bevel forms a straight line at a fixed angle to the shaft of the needle. The sharper the straight beveled needle tip, the smaller the angle of the bevel, resulting in a longer needle point. Conversely, if a shorter needle tip is required, then the angle of the bevel must be larger, resulting in a dull needle. However, in a concavely beveled needle tip 85, a bevel created such that the progressively steeper (less sharp) angle of the needle with increasing distance from the point permits maximum sharpness with minimal bevel length (i.e., the needle will enter the target vessel and almost immediately have its entire beveled portion within the vessel lumen). In other words, a progressive tip 85 allows an extremely sharp tip without the bevel taking up a larger part of the needle tip. This short distance of a concave bevel 85 is created by the non-linear shape of a concave bevel 85. The sharpness may allow safer access to the vessel/organ lumen because of a reduced possibility of the needle tip moving outside the vessel/organ lumen because its tip length is a smaller fraction of the lumen diameter than the tip of a standard long-bevel needle and because of its greater sharpness, there is less propability of double wall puncture of the target vessel/organ lumen. According to an embodiment of the disclosed invention, a progressive bevel can be created through the creation of bilateral curved secondary grinds or a combination of 3 or more bilateral straight grinds of progressively greater angle. FIG. 4 shows the placement of an o-ring 35 in the guide-wire leg 30 of Y-adapter 10, according to certain embodiments of the present invention. A bag 45 over a guide-wire 40 may have plugs 47 at the ends of bag 45 that may be attached to guide-wire leg 30 or sealed onto themselves through snaps 49. In some embodiments, plugs 47 may not seal onto themselves and may merely be attached to an additional cover. A guide-wire 40 within a bag 45 may have a straight tip on one side and a j-tip on another side. In the illustrated embodiment, a bag 45 may have plugs 47 on both sides such that the practitioner can choose to attach a straight tip side or a j-tip side of a guide-wire 40. Plugs 47 may connect to guide-wire leg 30 at a structure configured to hold an o-ring 35. The placement of o-ring 35 allows a seal such that contaminants and air during aspiration cannot enter bag 45. Accordingly, bag 45 may assist in preventing contaminants from entering a Y-adapter 10.

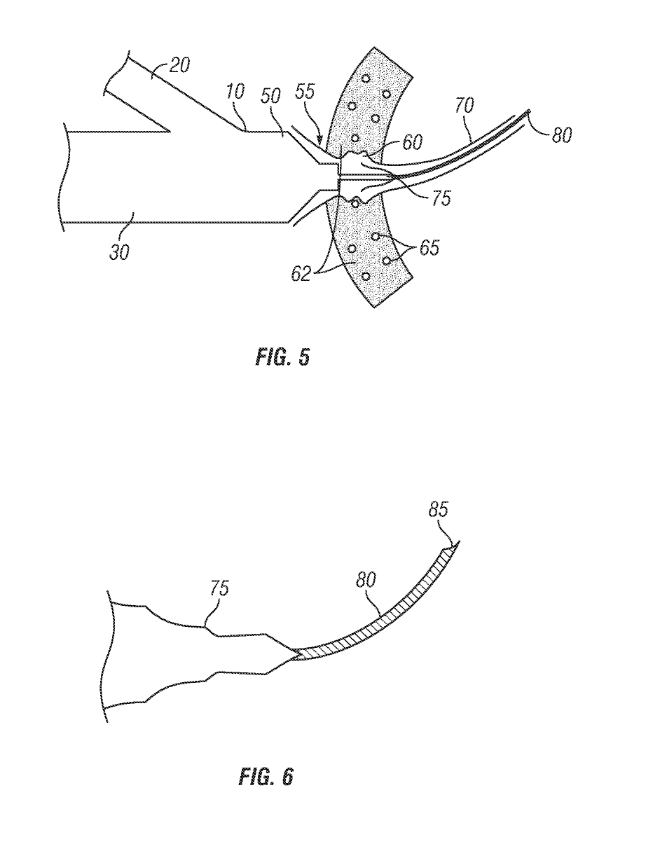

FIG. 5 shows the connection between front leg 50 Y-adapter 10 and a catheter hub 55 according to certain embodiments of the present invention. In some embodiments, front leg 50 may have a tapered configuration such that the end attaching to Y-adapter 10 is wider than the opposite end. In some embodiments, catheter hub 55 may be tightly fitted over the wider part front leg 50. A catheter hub may lead to a suture neck 60. In some embodiments, suture neck 60 may have removable attached wings, which may contain suture holes 65. In other embodiments suture neck 60 may have a removable attached elastomeric material 62, which may contain suture holes 65. As discussed above, elastomeric material 62 may prevent injury to the patient and prevent bacteria from entering the skin and/or injection site because of its close fit to the patient's skin. Suture neck 60 may connect or be bonded to a catheter 70. In some embodiments, a tapered needle hub 75 may fit tightly over the narrow part of front leg 50. Tapered needle hub 75 may lead into, and/or be bonded to, a cannulating needle 80.

FIG. 6 shows an internally tapering needle hub 75 which may attach to needle 80 according to certain embodiments of the present invention. Standard needle hubs that are not tapered have a 90 degree angle between the hub base and the needle shaft. With such a needle hub, it is very likely that a guide-wire attempting to pass through would become caught on the 90 degree bevel width reduction and not enter the needle lumen. Under a design utilizing a tapered needle hub 75 where there is a more gradual change in the needle hub's width, a guide-wire 40 may be much less likely to become caught.

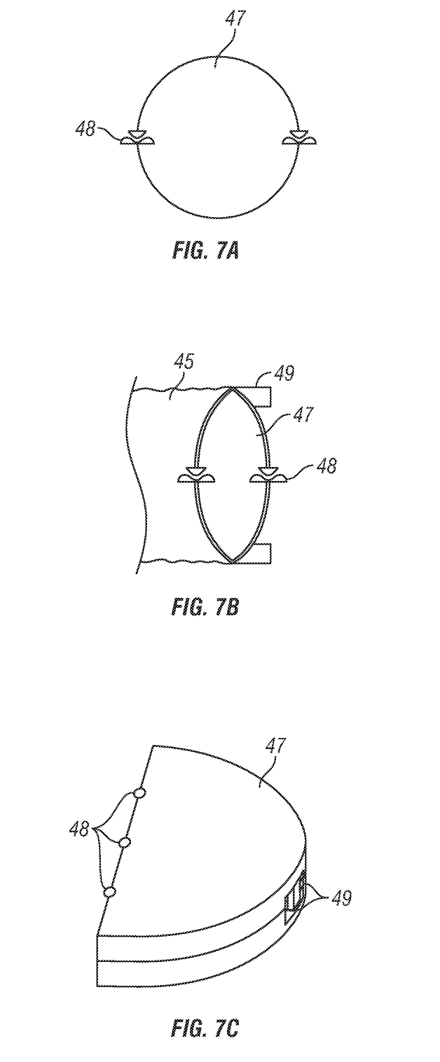

FIGS. 7A-C show a plug 47 configured to create an air- and water-tight seal with a cannulation device 5 at guide-wire leg 30. FIG. 7A shows a front view of a plug 47 that is bendable and/or hinged at connectors 48. FIG. 7B shows an angled view of a bag 45 leading to a plug 47 that is bendable at connectors 48 with snaps 49. FIG. 7C shows an angled view of a plug 47 bent at connectors 48 and sealed by snaps 49.

According to an exemplary embodiment of the disclosed method, a method of cannulating a vessel or cavity may include the steps of: providing device comprising a Y-adapter having three legs: a guide-wire leg, a syringe leg, and a front leg; providing a needle attached to the front leg of the device; inserting the needle into a desired vessel or cavity; pushing a guide-wire through the guide-wire leg of the Y-adapter, through the central cavity of the Y-adapter, and into the needle through the front leg of the Y-adapter such that the guide-wire enters the vessel or cavity; pushing the guide-wire to a desired location within the vessel or cavity; pushing a cannula or catheter over the guide-wire to the desired location within the vessel or cavity; and removing the guide wire and device.

In other embodiments, particularly when the pressure of the targeted vessel or cavity has sub-atmospheric pressure, the method may further include the steps of: providing a syringe; inserting said syringe into the syringe leg of the device; inserting the needle into a vessel or cavity; aspirating the syringe; and determining from the amount of force required for aspiration whether the vessel or cavity has been successfully entered. If the syringe is easily aspirated and fluid aspirated into the syringe barrel, then the cavity or vessel has likely been successfully entered. However, if a good deal of force is applied to withdrawing the syringe plunger with no fluid entering the syringe barrel, then it is likely that the vessel or cavity has not been successfully entered. In other embodiments, particularly where the targeted vessel or cavity has super-atmospheric pressure, these additional steps may not be desirable, as fluid from the vessel or cavity may automatically enter the needle thereby providing confirmation to the user that the vessel or cavity has been successfully entered.

In certain embodiments of the disclosed method, the guide-wire may have a straight-tip, a j-tip, or both (i.e. one each on opposite ends of the wire). A protective cover, such as a plastic bag, with the guide-wire contained therein, may also be provided to avoid the introduction of contaminants, such as from the user's hands, to the guide wire. In some embodiments, the cover/bag may include plugs on either or both ends where one plug may be connected to the guide-wire leg of the device, and the other end may be sealed either onto itself or by a separate cover. Thus, certain embodiments of the disclosed method may further include the steps of: connecting an end of the protective cover containing the guide-wire to the guide-wire leg of the device; gripping the cover over the guide-wire and advancing or retracting the wire into or out of the device; and gradually pulling back the bag as the wire is advanced such that the bag remains outside of the device as the guide-wire is pushed through the device as described above. In some embodiments wherein the guide-wire has both a straight and a j-tip, the user may, after selecting the desired tip, attached the side of the protective cover corresponding to that tip to the guide-wire leg and proceed as described above.

Certain embodiments of the disclosed method may further include the use of a catheter hub located over the front leg of the Y-adapter. After the cannula or catheter has been successfully placed, the catheter hub may be removed from the device. In some embodiments, the catheter or cannula my be sutured or taped to the skin of the patent through a suture neck and suture holes located on the catheter hub such that the catheter or cannula is held in place for an extended period of time. In other embodiments, the catheter hub may include, or even be comprised of, an elastomeric material. Said elastomeric material may be flexed to conform to the shape of the patient's skin at the injection site and then taped or sutured in place. In other embodiments, the catheter hub may further include a fluid line adapter.

Although this disclosure has been described in terms of certain embodiments and generally associated methods, alterations and permutations of these embodiments and methods will be apparent to those skilled in the art. For example, it will be understood that the disclosed methods are for illustration purposes only and that the described or similar techniques may be performed at any appropriate time, including concurrently, individually, or in combination. In addition, many of the described steps may take place simultaneously and/or in different orders than as shown. Accordingly, the above description of example embodiments does not define or constrain this disclosure. Other changes, substitutions, and alterations are also possible without departing from the spirit and scope of this disclosure.

LIST FOR REFERENCE NUMERALS

| (Part No.) | (Description) |
|---|---|
| 5 | cannulation device |
| 10 | Y-adapter |
| 15 | central cavity of Y-adapter |
| 20 | syringe leg of Y-adapter |
| 25 | syringe |
| 30 | guide-wire leg of Y-adapter |
| 32 | wire-guide |
| 35 | o-ring |
| 40 | guide-wire |
| 45 | protective cover/bag over guide-wire |
| 47 | plug |
| 49 | snap |
| 50 | front leg of Y-adapter |
| 55 | catheter hub |
| 60 | suture neck |
| 62 | elastomeric material |
| 65 | suture holes |
| 70 | catheter |
| 75 | tapered hub |
| 80 | needle |
| 85 | progressively beveled needle tip |
| 90 | straight beveled needle tip |

The following U.S. patents are incorporated herein by reference:

U.S. Pat. No. 4,143,853—Valve for use with a catheter or the like
U.S. Pat. No. 4,387,879—Self-sealing connector for use with plastic cannulas and vessel catheters
U.S. Pat. No. 4,535,819—Valve assembly
U.S. Pat. No. 4,828,549—Over-the-needle catheter assembly
U.S. Pat. No. 4,850,960—Diagonally tapered, beveled tip introducing catheter and sheath and method for insertion
U.S. Pat. No. 4,935,008—Double lumen introducing needle
U.S. Pat. No. 5,092,846—Introducer for medical tube
U.S. Pat. No. 5,284,476—Nuclear Hydrolysis Cannula
U.S. Pat. No. 5,358,490—Apparatus for use in central vein cannulation
U.S. Pat. No. 6,015,401—Methods for Vessel Cannulation
U.S. Pat. No. 7,063,685—Hemostasis valve for a catheter

What is claimed is:

1. An apparatus for cannulation, comprising:
a Y-adapter, having:
a central cavity;
a front leg;
a guide-wire leg; and
a syringe leg;
a needle;
a syringe;
a wire guide; and
a guide-wire,
wherein the guide-wire leg is positioned opposite the central cavity from, and in-line with, the front leg;
wherein the front leg is configured to hold the needle such that the needle is pointing away from the apparatus;
wherein the syringe leg is configured to receive the syringe;
wherein the syringe is configured for aspiration; and
wherein the wire guide is positioned inside the guide-wire leg such that the guide-wire leg is configured to receive the guide-wire and to guide it through the central cavity and into the needle via the front leg.

2. The apparatus of claim 1, wherein the syringe leg is parallel to the guide-wire leg.

3. The apparatus of claim 1, wherein the syringe leg is at an angle from about zero (0) degrees to about ninety (90) degrees to the guide wire leg.

4. The apparatus of claim 1, wherein the guide-wire has at least one of a straight tip and a j-tip.

5. The apparatus of claim 1, further comprising a protective cover having a first end and a second end, wherein the protective cover is positioned around the guide-wire and configured to protect the guide-wire from contaminants as it is pushed into the guide-wire leg.

6. The apparatus of claim 5, wherein the protective cover is a plastic bag.

7. The apparatus of claim 5, further comprising at least one plug, wherein the at least one plug is positioned at least one of the first end of the protective cover and the second end of the protective cover.

8. The apparatus of claim 7, wherein the at least one plug is configured to attach to the guide-wire leg and to allow the guide-wire within the protective cover to be pushed into the guide-wire leg.

9. The apparatus of claim 8, further comprising an o-ring, wherein the o-ring is configured to act as a seal between the at least one plug and the guide-wire leg.

10. The apparatus of claim 1, wherein the shape of the needle is one of straight and curved.

11. The apparatus of claim 10, further comprising a valve, wherein the valve is configured to prevent backflow of fluids into at least one of the wire-guide and the guide-wire leg.

12. The apparatus of claim 1, further comprising a catheter hub, wherein the catheter hub comprises:
a suture neck having at least one suture hole; and
a catheter,
wherein the catheter hub is configured to attach to the patient during cannulation;
wherein the suture neck contains the catheter;
wherein the catheter hub is positioned at the exterior end of the front leg; and
wherein the catheter hub covers a portion of the needle.

13. The apparatus of claim 12, wherein the catheter hub is configured to attach to the patient by at least one of suturing and taping.

14. The apparatus of claim 12, further comprising a set of extended wings having at least one suture hole, wherein the set of extended wings is connected to the suture neck and configured to attach to the patient by at least one of suturing and taping.

15. The apparatus of claim 14, wherein at least one of the suture neck and the extended wings includes an elastomeric material.

16. The apparatus of claim 12, further comprising a tapered needle hub, wherein the needle hub is configured to guide a guide-wire from the catheter hub into the needle such that the guide wire does not snag as it advances from the catheter hub to the needle.

17. The apparatus of claim 1, wherein the needle is one of a straight beveled needle and a concavely beveled needle.

18. The apparatus of claim 1, wherein the front leg is tapered.

19. A method for cannulating a vessel or cavity, comprising:
providing a device for cannulation comprising a front leg, a guide wire leg opposite the front leg and configured to receive a guide-wire, a syringe leg configured to receive a syringe, and a needle, wherein the needle is connected to the front leg;
inserting the needle into a desired vessel or cavity;
pushing a guide-wire through the guide-wire leg and into the needle through the front leg of the device;
advancing the guide wire to a desired location within the vessel or cavity;
providing at least one of a cannula and a catheter;
pushing the at least one of a cannula and a catheter over the guide-wire to the desired location within the vessel or cavity;
removing the guide-wire and the device.

20. The method of claim 19, further comprising the steps of:
prior to the step of pushing a guide-wire through the guide-wire leg:
providing a syringe;
placing the syringe into the syringe leg; and
aspirating the syringe to verify that the desired vessel or cavity has been successfully entered.

21. The method of claim 19, further comprising the steps of:
providing a protective covering, wherein the guide wire is initially contained within the protective covering; and
as the guide-wire is pushed into the guide-wire leg; gripping the protective cover such that the cover remains outside of the device as the guide-wire is advanced.

22. The method of claim 19, wherein the guide-wire has at least one of a straight tip and a j-tip.

23. An apparatus for cannulation, comprising:
a Y-adapter, having:
a central cavity;
a front leg;
a guide-wire leg; and
a syringe leg;
a needle;
a syringe;
a wire guide; and
a guide-wire,
wherein the needle is one of a straight beveled needle and a concavely beveled needle.

24. The apparatus of claim 23, wherein the guide-wire leg is positioned opposite the central cavity from, and in-line with, the front leg.

25. The apparatus of claim 24, wherein the front leg is configured to hold the needle such that the needle is pointing away from the apparatus; wherein the syringe leg is configured to receive the syringe; wherein the syringe is configured for aspiration; and wherein the wire guide is positioned inside the guide-wire leg such that the guide-wire leg is configured to receive the guide-wire and to guide it through the central cavity and into the needle via the front leg
